(19) United States
(12) Patent Application Publication
(10) Pub. No.: US 2013/0158356 A1
(43) Pub. Date: Jun. 20, 2013

(54) LARYNGOSCOPE

(71) Applicant: Taner Yilmaz, Ankara (TR)

(72) Inventor: Taner Yilmaz, Ankara (TR)

(21) Appl. No.: 13/814,733

(22) PCT Filed: Aug. 22, 2011
(86) PCT No.: PCT/IB2011/053670

§ 371 Date: Feb. 7, 2013

(30) Foreign Application Priority Data
Sep. 2, 2010 (TR) 2010/07321

(51) Int. Cl. A61B 1/267; A61B 1/06; A61M 16/04; A61B 1/00

(52) U.S. Cl. CPC A61B 1/267; A61B 1/00094; A61B 1/0676; A61M 16/0488
USPC 600/187

(57) ABSTRACT

This invention is related to a laryngoscope used in endoscopic surgery of the larynx. The laryngoscope (1) which is the subject of this invention includes; a body (2) which is cylinder in shape and empty inside with one end placed inside the larynx, at least one handle (6) fixed with one tip to the other end of the body (2) and at least one sleeve (7) which is fixed to the other end of the handle (6) unconnected to the body (2) and facilitates the motion of the body (2). The body (2) includes; at least a canal (3)upon which the intubation tube is placed; a ridge (4)preventing the slippage of the intubation tube within the larynx; and at least two burrows (5) located on its end attached to the handle (6).

### TECHNICAL AREA

This invention is related to a laryngoscope used in endoscopic surgery of the larynx.

### PREVIOUS TECHNIQUE

In the known technique of endoscopic surgery of the larynx, a laryngoscope is used to observe the lumen of the larynx. an intubation tube is placed inside the larynx to enable the patient's respiration during surgical intervention. This intubation tube remains outside and below the laryngoscope. In case of surgical interventions to the posterior half of the larynx, however, the intubation tube must be elevated anteriorly with the laryngoscope. During surgical intervention the intubation tube must stay above the laryngoscope. Because both intubation tube and laryngoscope are cylindirical in shape and because there is no structure to fix the intubation tube on the laryngoscope, the intubation tube slides from the top of laryngoscope and may fall to the posterior part of the larynx. In such a case, the operation must be paused and the intubation tube must be replaced again above the laryngoscope. Aforementioned procedures prolong the operation and sometimes even prevent the accomplishment of the operation.

In the known technique, United States patent document numbered U.S. Pat. No. 5,665,052 mentions about a laryngoscope and a multifunctional intubation stilet used during endoscopic procedure.

In another application of the known technique, international patent document numbered WO2006118984 mentions about a laryngoscope carrying intubation tube and a flexible imaging equipment. Because the intubation tube is connected to the laryngoscope it both gives a chance to the surgeon to inspect the larynx of the patient, and facilitates the surgeon's job in patients whose endoscopic procedures will be difficult to perform.

### SHORT DESCRIPTION OF THE INVENTION

The aim of this invention is to produce a laryngoscope which prevents the slipping of the intubation tube from above the laryngoscope during surgical procedures within the larynx.

Another aim of this invention is to produce a laryngoscope which enables the accomplishment of a surgical procedure to the posterior part of the larynx in a shorter time.

### DETAILED DESCRIPTION OF THE INVENTION

A laryngoscope that is produced to accomplish the goal of this invention is shown in the attached figures. From these figures

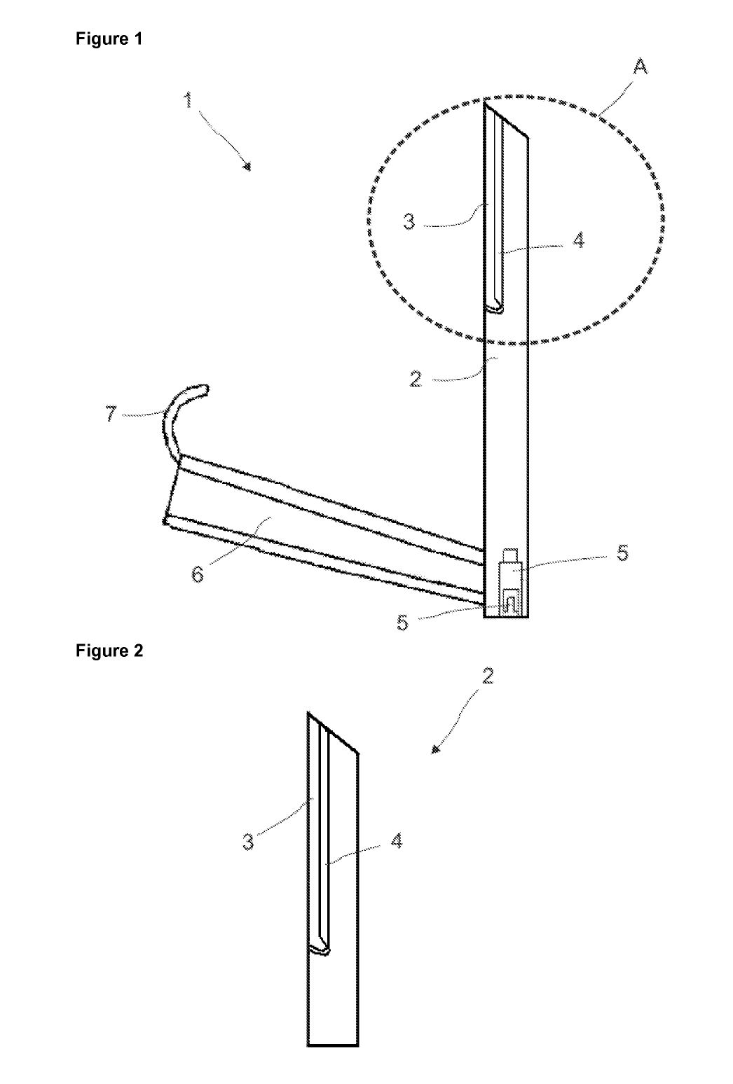

FIG. 1 is the side view of the laryngoscope which is the subject of this invention.

FIG. 2 is the sectional view of the “A” detail of the laryngoscope which is the subject of this invention.

The parts in the figures were numbered one by one, and their equivalents were given below.

1. Laryngoscope

2. Body

3. Canal

4. Ridge

5. Burrow

6. Handle

7. Sleeve

The laryngoscope (1) which is the subject of this invention includes;
a body (2) which is cylinder in shape and empty inside with one end placed inside the larynx.
at least one handle (6) fixed with one tip to the other end of the body (2) and
at least one sleeve (7) which is fixed to the other end of the handle (6) unconnected to the body (2) and facilitates the motion of the body (2).

The body (2) includes; at least a canal (3) which runs along the upper and outer wall of the body (2) starting from its tip placed within the larynx towards its end having the handle (6) and upon which the intubation tube is placed; at least one ridge (4) running along the outer side of the canal (3) and preventing the slippage of the intubation tube within the larynx; and at least two burrows (5) located on its end attached to the handle (6).

The canal (3) is as large as a portion of the surface of the intubation tube can lie upon, and can be located on the left, right or midline of the lateral wall of the body (2) depending on usage. In the preferred usage of the invention the canal (3) is semicircle in shape.

The ridge (4) runs along the canal (3) and is as long as the canal (3). The most superior end of the ridge (4) is on a level with the most superior end of the body (2) placed within the larynx. The ridge (4) fixes the laryngeal part of the intubation tube placed within the canal (3) and prevents the slippage of the intubation tube from over the body (2).

For the user to accomplish the surgical procedure comfortably, a light source is placed within one of the burrows (5) and aspirator cannula which functions to create vacuum is placed within the other burrow (5).

The handle (6) is used by the user to insert the laryngoscope (1) easily into the place where he would perform the surgical procedure easily, and to be able to change the direction of laryngoscope (1) in its place where the surgical procedure is carried out. performed. The sleeve (7), being structured for the user to introduce his finger, enables the user to move the laryngoscope (1) in vertical plane.

By introducing his fingers through the sleeve (7), the user grasps the handle (6) and inserts the body (2) inside the larynx. The user accomplishes the procedure easily because he can comfortably see inside the larynx with the help of light attached to the burrow (5) on the body (2). While the user is performing the aforesaid surgical operation, the intubation tube lies fixed on top of the body (2) and the problems such as slipping of the intubation tube are abolished. Thereby, the surgical procedure inside the larynx is accomplished easily.

The development of a wide variety of applications of the laryngoscope (1) which is the subject of this invention are possible. The invention cannot be restricted to the examples that are explained here. Basically it is as specified in the claims.

1-6. (canceled)

7. A laryngoscope comprising: a body in cylinder shape with empty inside with the tip adapted to be placed within the larynx; at least one handle fixed with one tip to the other end of the body; at least one sleeve which is fixed to the other end of the handle unconnected to the body and facilitates the motion of the body; at least one canal which runs along the outer wall of the body starting from its tip placed within the larynx towards its end having the handle and upon which the intubation tube is placed; a ridge running along the outer side of the canal and preventing the slippage of the intubation tube within the larynx; and at least two burrows located on its end attached to the handle and being adapted for placement of a light source and aspiration cannula within.

8. The laryngoscope of claim 7 wherein the canal is as large as a portion of the surface of an intubation tube can lie upon; and wherein the canal can be located on the left, right or midline of the lateral wall of the body depending on usage.

9. The laryngoscope of claim 7 wherein the canal is semicircular in shape.

10. The laryngoscope of claim 8 wherein the canal is semicircular in shape

11. The laryngoscope of claim 7 further comprising a ridge wherein the ridge runs along the canal and the ridge is as long as the canal.

12. The laryngoscope of claim 8 further comprising a ridge wherein the ridge runs along the canal and the ridge is as long as the canal.

13. The laryngoscope of claim 9 further comprising a ridge wherein the ridge runs along the canal and the ridge is as long as the canal.

14. The laryngoscope of claim 10 further comprising a ridge wherein the ridge runs along the canal and the ridge is as long as the canal.

15. A laryngoscope of any claims 11 to 14 wherein the most superior end of the ridge is on a level with the most superior end of the body placed within the larynx.

16. A laryngoscope of any claims 11 to 14 wherein the ridge fixes the laryngeal part of the intubation tube placed within the canal and which prevents the slippage of the intubation tube from over the body.
